(19) United States
(12) Patent Application Publication
(10) Pub. No.: US 2012/0083627 A1
(43) Pub. Date: Apr. 5, 2012

(54) Method of Synthesis of Arylsulfur Trifluorides and Use as in situ Deoxofluorination Reagent

(75) Inventors: William R. Dolbier, JR., Gainesville, FL (US); Wei Xu, Gainesville, FL (US)

(73) Assignees: FLUOROTECH, LLC, Gainesville, FL (US); OAKWOOD PRODUCTS, INC., West Columbia, SC (US)

(21) Appl. No.: 13/249,385

(22) Filed: Sep. 30, 2011

Related U.S. Application Data
(60) Provisional application No. 61/388,660, filed on Oct. 1, 2010.

(51) Int. Cl. C07C 381/00

(52) U.S. Cl. 568/74

(57) ABSTRACT

The invention is a method of synthesizing Arylsulfur Trifluorides, such as Fluolead, by reacting BR₂ and KF (or suitable alkali metal fluoride) in acetonitrile (or other suitable solvent). The invention also comprises using the Fluolead (or its substitutes), thus prepared, in situ as deoxofluorination reagents with a suitable aldehyde, ketone, or alcohol such as one selected from the group consisting of benzaldehyde Benzaldehyde, p-Bromobenzaldehyde, p-Tolualdehyde, Acetophenone, 2-Butanone, or Isobutyraldehyde, wherein the mixture is heated to reflux until completion. The respective products are then isolated after extraction by hexane and destruction of the sulfinyl fluoride co-product.

### RELATED APPLICATIONS

This application claims the benefit of U.S. Application 61/388,660 filed Oct. 1, 2010.

### FEDERALLY SPONSORED RESEARCH OR DEVELOPMENT

Not applicable

### REFERENCED OR INCORPORATED MATERIAL

Not applicable.

### FIELD OF INVENTION

The present invention relates to deoxofluorinating agents, such as arylsulfur trifluorides, and more particularly to a novel method of synthesis of arylsulfur trifluorides and their in situ use.

### BACKGROUND OF THE INVENTION

Selective incorporation of fluorine into organic molecules continues to be an important and ever-challenging component in the design and synthesis of effective pharmaceuticals and agrochemicals. Considering the various techniques that are utilized to accomplish such incorporation, deoxofluorination reactions certainly must be considered among the most important. Deoxofluorination reactions include most notably the direct conversion of alcohols to alkyl fluorides, ketones and aldehydes to gem-difluoroalkanes and carboxylic acids to trifluoromethyl groups.

It may be said that the discovery that SF₄ could act as an effective reagent for carrying out such transformations, most useful for the fatter two, was a key factor in propelling the emerging field of synthetic organofluorine chemistry into the main stream of synthetic organic chemistry during the 1960's. In the ensuing years other reagents, essentially derivatives of SF₄, emerged as safer, more convenient, and sometimes superior deoxofluorination reagents. The best known, and most widely used among them are DAST (diethylaminosulfur trifluoride) and Deoxo-Fluor (bis(2-methoxyethyl)aminosulfur trifluoride), although there are many others that have more limited applicability.

Recently, two other broadly effective deoxofluorination reagents have been reported: Xtal-Fluor (the E-version being derived from DAST), and Fluolead, a crystalline, highly reactive arylsulfur trifluoride. Such Fluolead reagents are described by Umemoto in U.S. Pat. No. 7,265,247 (issued Sep. 4, 2007), U.S. Pat. No. 7,501,543 (issued Mar. 10, 2009), and US Patent Publication Number 2009/0203924 A1 (published Aug. 13, 2009). Interestingly, all of these broadly effective, diverse deoxofluorination reagents are sulfur fluoride compounds.

Looking at Fluolead, as a thermally stable, crystalline deoxofluorination agent, its broad and highly efficient reactivity with alcohols, aldehydes, ketones and carboxylic acids can be exemplified in Scheme 1.

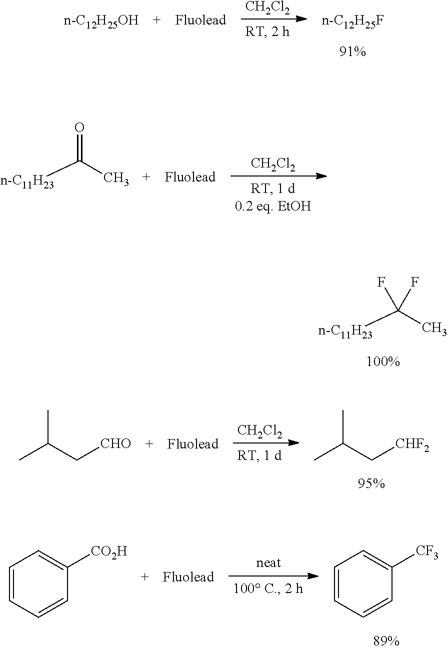

As Umemoto has described in '247, '543, and '924 referenced above, Fluolead can be prepared by treating an acetonitrile solution of precursor disulfide, with 3.5 equivalents of chlorine in the presence of 4 equivalents of anhydrous KF. The Cl₂ was slowly bubbled into the ice-bath-cooled solution over a period of two hours. After filtration and distillation, a yield of 55% of Fluolead could be obtained.

The toxicity of gaseous Cl₂ and control of its addition to the reaction mixture can pose practical problems in the laboratory synthesis of Fluolead, as can the unavoidable partial over-chlorination of Fluolead to form chlorotetrafluorosulfur. Though the partial over-chlorination of Fluolead to form chlorotetrafluorosulfur is useful for further conversion to the analogous aryl-SF₅ compound, as shown by U.S. Pat. No. 7,592,491 to Umemoto, it is, nevertheless, problematic if one desires to obtain high yields of arylsulfur trifluorides, such as Fluolead.

Thus, there exists a need in the art to obtain high yields of Arylsulfur Trifluorides without the requirement to resort to the use of toxic gaseous Cl₂.

Further, although arylsulfur trifluoride compounds, in particular Fluolead, are outstanding deoxofluorination reagents, with broad applicability, there are problems associated with their preparation, storage and use. Fluolead, like all other arylsulfur trifluorides, is corrosive to glass and moisture sensitive. The major impurity in samples of Fluolead, and one that is virtually impossible to totally eradicate, is the respective arylsulfinyl fluoride compound that is derived from the reaction of the Fluolead SF₃ group with traces of water in the reaction mixture. With that in mind, effort needs to be made to use the driest possible KF in the preparation of Fluolead. This sulfinyl fluoride is also the co-product from Fluolead in its deoxofluorination reactions, and it is itself corrosive to glass. Thus Fluolead must be stored in flasks constructed of fluoropolymer, and reactions of Fluolead are best carried out in fluoropolymer bottles or flasks.

Thus, there exists a need in the art to develop a method of using Fluolead wherein such storage problems and moisture sensitivity issues are reduced.

### SUMMARY OF THE INVENTION

The present invention provides a novel method of synthesizing Arylsulfur Trifluorides and combining that method for use as in situ Deoxofluorination Reagent.

It is thus an object of the present invention to provide a method of synthesizing Arylsulfur Trifluorides without the requirement to resort to the use of toxic gaseous Cl₂. To this end, a method of producing Arylsulfur Trifluorides without the use of Cl₂ is herein disclosed. This method essentially replaces the Cl₂ and KF reaction with a Br₂ and KF reaction (or other suitable alkali metal fluoride). In this way, when Disulfide is allowed to react with excess Br₂ and dry, excess KF in acetonitrile (or some other suitable polar arprotic solvent), Fluolead is obtained in 85% yield, the reaction being carried out for two hours at 0° C. followed by four hours at room temperature.

It is a second object of the present invention to provide a method whereby the storage issues associated with Fluolead's reactivity with glass and susceptibility to water are reduced. To this end, in situ use of Arylsulfur Trifluorides as Deoxofluorination Reagent is disclosed. In this way, the acetonitrile reaction mixture containing prepared Fluolead can be used directly to carry out its reactions with alcohols, aldehydes, or ketones. The alcohols, aldehydes, or ketones are added to the acetonitrile reaction mixture containing prepared Fluolead and then the mixture must be heated at reflux. The products can then be isolated after extraction by hexane and destruction of the sulfinyl fluoride co-product by treatment with 10% aq. NaOH.

It is a third object of the present invention to provide a method for in situ Deoxofluorination that substitutes simpler arylsulfur trifluorides for Fluolead. To this end, a method of substituting the mesityl reagent (2,4,6-trimethylphenylsulfur trifluoride) for in situ Deoxofluorination in place of Fluolead is disclosed. Also to this end, a method of substituting 2,6-dimethylphenyl disulfide is also disclosed.

### BRIEF DESCRIPTION OF THE SEVERAL VIEWS OF THE DRAWINGS

FIG. 1 is an example of the process for synthesis of Fluolead using Br₂ and KF in acetonitrile.

FIG. 2 is an example of the process for the in situ reaction with Fluolead.

FIG. 3 is a Table showing in situ generation and use of Fluolead.

FIG. 4 is an example of the processes of further in situ reactions using other disulfides.

FIG. 5 is an example of the processes of further in situ reactions using various arylsulfur trifluorides.

FIG. 6 is formula (I) and formula (Ia).

FIG. 7 is formula (II) and formula (IIa).

### DETAILED DESCRIPTION

It is to be understood by a person having ordinary skill in the art that the present discussion is a description of exemplary embodiments only and is not intended as limiting the broader aspects of the present invention. The following example is provided to further illustrate the invention and is not to be construed to unduly limit the scope of the invention.

The present invention provides a novel method of synthesizing Arylsulfur Trifluorides and combining that method for use as in situ Deoxofluorination Reagent.

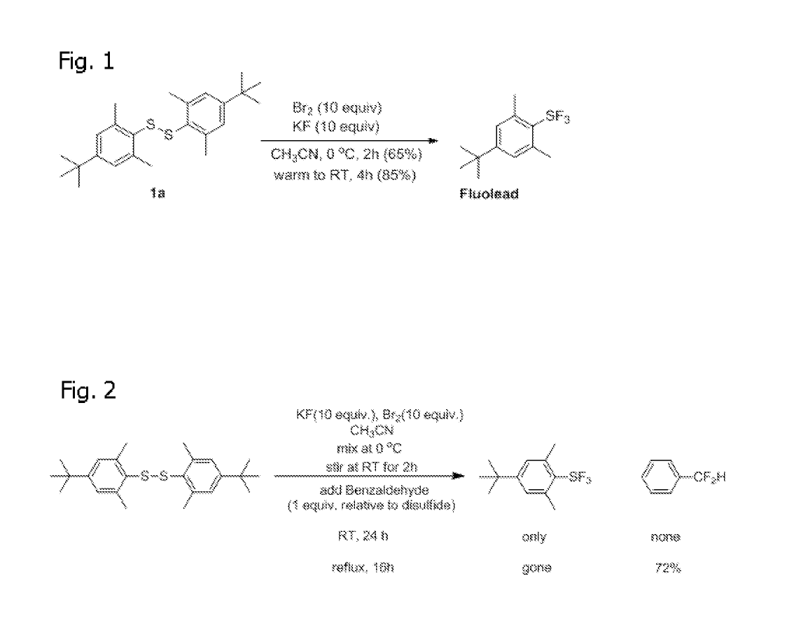

As summarized above, synthesis of Arylsulfur Trifluorides, such as Fluolead, is achieved by allowing the disulfide to react with excess Br₂ and dry, excess KF (or another suitable dried alkali metal fluoride) in acetonitrile (or a suitable solvent such as a polar arprotic solvent) where the reaction is carried out for two hours at 0° C. followed by four hours at room temperature. See FIG. 1. Moreover, three equivalents of Br₂ and six equivalents of KF are sufficient to convert all of an aryl disulfide to the arylsulfur trifluoride.

That is, formula (I), is prepared by reacting a compound of formula (Ia) with a quantity of Bromine and an alkali metal fluoride, in which: R_{1α} and R_{1β} are independently a hydrogen atom or a primary or secondary alkyl group having from one to eight carbon atoms; R_{2α} and R_{2β} are independently a hydrogen atom or a primary, secondary, or tertiary alkyl group having from one to eight carbon atoms; R₃ is a hydrogen atom, a halogen atom, or a primary, secondary, or tertiary alkyl group having from one to eight carbon atoms; wherein, when R₃ is a hydrogen atom, at least two of R_{1α}, R_{1β}, R_{2α}, and R_{2β} are primary, secondary, or tertiary alkyl groups having from one to eight carbon atoms and the others are a hydrogen atom, and wherein, when R₃ is a primary alkyl group having from one to eight carbon atoms, at least one of R_{1α}, R_{1β}, R_{2α}, and R_{2β} is a primary, secondary, or tertiary alkyl group having from one to eight carbon atoms and the others are a hydrogen atom, and wherein when at least two of R_{2α}, R_{2β}, and R₃ are tertiary alkyl groups, the tertiary alkyl groups are non-adjacent. See FIG. 6.

Similarly, formula (II) is prepared by reacting a compound of formula (IIa) with a quantity of Bromine and an alkali metal fluoride, in which: R_{1α}, and R_{1β} are independently a hydrogen atom or a primary or secondary alkyl group having from one to eight carbon atoms; R₃ is a hydrogen atom, a halogen atom, or a primary, secondary, or tertiary alkyl group having from one to eight carbon atoms; wherein when R₃ is a hydrogen atom, R_{1α} and R_{1β} are independently a primary or secondary alkyl group having from one to eight carbon atoms, and wherein, when R₃ is a primary alkyl group having one to eight carbon atoms, at least one of R_{1α} and R_{1β} is a primary or secondary alkyl group having from one to eight carbon atoms and the other is a hydrogen atom. See FIG. 7.

Specifically, by way of but one example, the preparation of 2,6-dimethyl-4-t-butylphenylsulfur trifluoride (Fluolead) can be carried out as follows: into a flame-dried 500 mL round-bottomed flask equipped with a dropping funnel, place anhydrous acetonitrile (120 mL) under nitrogen. Spray-dried potassium fluoride (29.1 g, 500 mmol) is added with stirring, followed by adding bis(2,6-dimethyl-4-t-butylphenyl)disulfide (19.4 g, 50 mmol). The mixture is then cooled to 0° C. by an ice bath, and bromine (26 mL, 500 mmol) is added dropwise. After addition, the mixture is stirred at 0° C. for 2 hours. The solvent and excess of bromine are removed under reduced pressure at room temperature. After that, the product is distilled under reduced pressure to give 17.5 g of a pale white solid (70% yield). ¹⁹FNMR (d-CH₃CN): δ 50.9 (t, J=62.7 Hz, 2F), −58.4 (d, J=62.7 Hz, 1F).

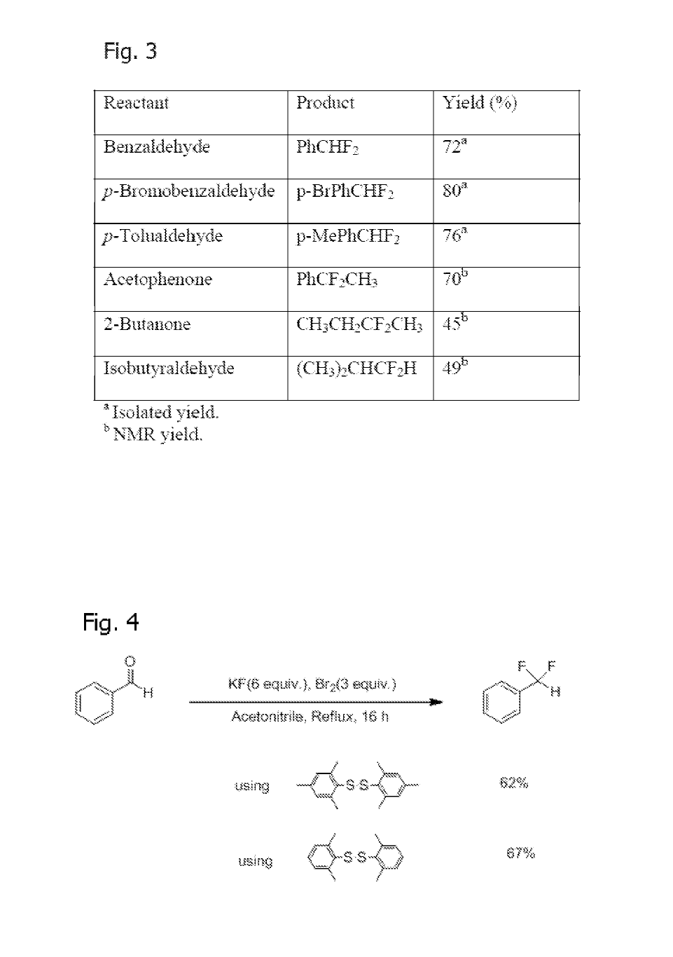

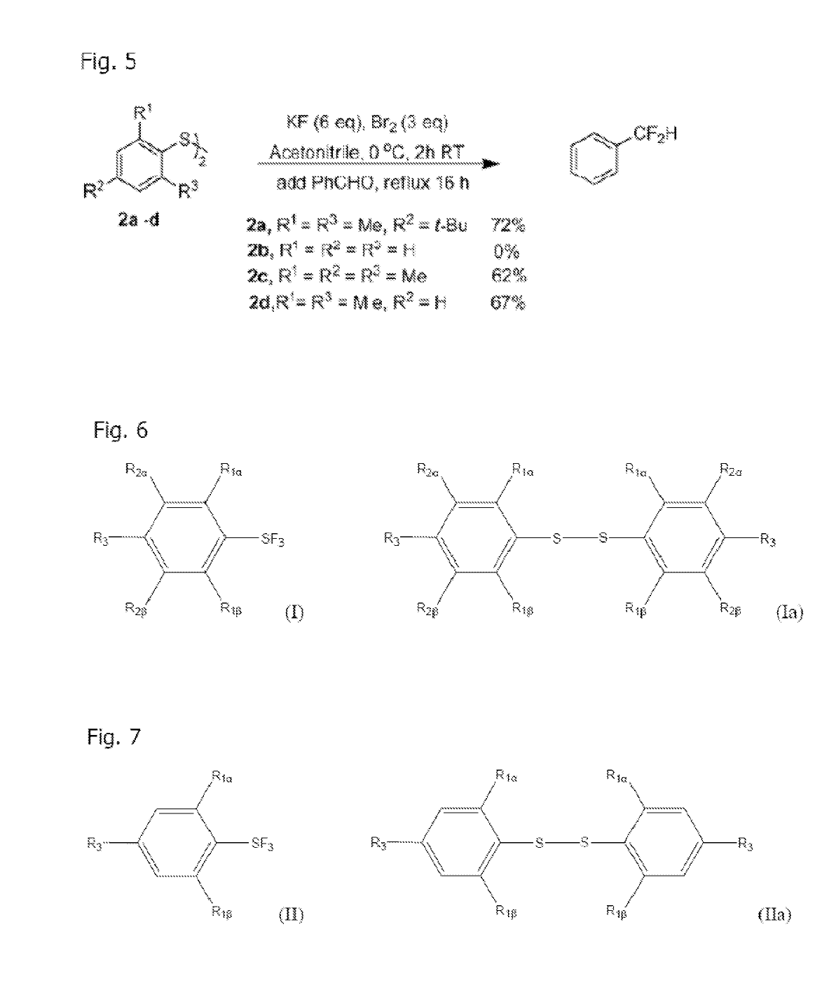

As summarized above, the in situ deoxofluorination procedure is carried out by adding the suitable alcohol, aldehyde, or ketone (such as but not limited to: Benzaldehyde, p-Bromobenzaldehyde, p-Tolualdehyde, Acetophenone, 2-Butanone, or Isobutyraldehyde) to the acetonitrile reaction mixture containing prepared Fluolead (or a substituted mesityl SF₃ etc. . . . as shown in FIGS. 4 and 5) and then the mixture must be heated at reflux for 16 hours (or until completion). FIG. 2 shows this reaction. The products can then be isolated after extraction by hexane and destruction of the sulfinyl fluoride co-product by treatment with 10% aq. NaOH. FIG. 3 shows these products from this in situ use of Fluolead.

The in situ process is exemplified by the following general procedure: All glassware was flame dried and cooled under nitrogen; potassium fluoride was dried at 140° C. under vacuum. To a 250 mL three-necked flask was added potassium fluoride (8.71 g, 150 mmol), disulfide (9.67 g, 25 mmol) and anhydrous acetonitrile (50 mL) under nitrogen. The mixture was cooled to 0° C., and bromine (12 g, 75 mmol) was added drop wise in 15 minutes. After addition, the cold bath was removed, and the reaction was stirred at RT for 2 h. Then the aldehyde (one equivalent relative to disulfide) was added in one portion. The mixture was heated to reflux and stirred for 16 h. cooled to RT, hexane (100 mL) added, followed by water (50 mL), and the upper layer was separated and washed with brine. Then this hexane extract was stirred with 10% sodium hydroxide (50 mL) for 1 hour. The upper layer was isolated and dried over sodium sulfate, and the product was purified with column or by distillation.

What is claimed is:

1. A method of preparing 2,6-dimethyl-4-t-butylphenylsulfur trifluoride comprising the steps of:
a. first providing a quantity of suitable solvent, an excess quantity of dried alkali metal fluoride, a quantity of bis(2,6-dimethyl-4-t-butylphenyl)disulfide, and an excess quantity of bromine;
b. allowing said quantity of bis(2,6-dimethyl-4-t-butylphenyl)disulfide to react with said excess quantity of bromine and said excess quantity of dried alkali metal fluoride in said quantity of suitable solvent.

2. The method of claim 1 wherein said excess quantity of dried alkali metal fluoride is a dried potassium fluoride and wherein said suitable solvent is a polar arprotic solvent.

3. The method of claim 1 further comprising the additional steps of:
c. allowing the reaction of step b to proceed for a two hour period at 0° C.
d. allowing the reaction of step b to proceed for an additional four hour period at room temperature.

4. The method of claim 2 wherein said excess quantity of dried potassium fluoride, said quantity of bis(2,6-dimethyl-4-t-butylphenyl)disulfide, and said excess quantity of bromine, are, respectively, six equivalents of dried potassium fluoride, one equivalent of bis(2,6-dimethyl-4-t-butylphenyl)disulfide, and three equivalents of bromine.

5. The method of claim 4 wherein the reaction of step b comprises the sub-steps of: first, placing said quantity of suitable solvent under nitrogen, second, stirring in said excess quantity of dried potassium fluoride, third, adding said quantity of bis(2,6-dimethyl-4-t-butylphenyl)disulfide, fourth, cooling the mixture to 0° C., and fifth, adding said bromine, dropwise.

6. The method of claim 5 wherein said polar arprotic solvent is anhydrous acetonitrile.

7. A method of using 2,6-dimethyl-4-t-butylphenylsulfur trifluoride as in situ deoxofluorination reagent comprising the steps of:
a. first providing a quantity of suitable solvent, an excess quantity of dried alkali metal fluoride, a quantity of bis(2,6-dimethyl-4-t-butylphenyl)disulfide, an excess quantity of bromine, and a quantity of suitable aldehyde or ketone;
b. allowing said quantity of bis(2,6-dimethyl-4-t-butylphenyl)disulfide to react with said excess quantity of bromine and said excess quantity of dried alkali metal fluoride in said quantity of suitable solvent;
c. allowing the reaction of step b to proceed for a two hour period at room temperature;
d. adding said quantity of aldehyde or ketone;
e. heating mixture to reflux until completion.

8. The method of claim 6 wherein said suitable solvent is a polar arprotic solvent and wherein said quantity of dried alkali metal fluoride is dried potassium fluoride.

9. The method of claim 8 wherein said excess quantity of dried potassium fluoride, said quantity of bis(2,6-dimethyl-4-t-butylphenyl)disulfide, said excess quantity of bromine, and said quantity of aldehyde or ketone are, respectively, six equivalents of dried potassium fluoride, one equivalent of bis(2,6-dimethyl-4-t-butylphenyl)disulfide, three equivalents of bromine, and 0.75 equivalents of aldehyde or ketone.

10. The method of claim 9 wherein said quantity of aldehyde or ketone is selected from the group consisting of: Benzaldehyde, p-Bromobenzaldehyde, p-Tolualdehyde, Acetophenone, 2-Butanone, or Isobutyraldehyde.

11. The method of claim 10 wherein the reaction of step b comprises the sub-steps of: first, placing said quantity of anhydrous acetonitrile under nitrogen, second, stirring in said excess quantity of dried potassium fluoride, third, adding said quantity of bis(2,6-dimethyl-4-t-butylphenyl)disulfide, fourth, cooling the mixture to 0° C., and fifth, adding said bromine, dropwise.

12. A method of using 2,4,6-trimethylphenylsulfur trifluoride as in situ deoxofluorination reagent comprising the steps of:
a. first providing a quantity of suitable solvent, an excess quantity of dried alkali metalfluoride, a quantity of mesityl disulfide, an excess quantity of bromine, and a quantity of suitable substrate for deoxofluorination;
b. allowing said quantity of mesityl disulfide to react with said excess quantity of bromine and said excess quantity of dried alkali metal fluoride in said quantity of suitable solvent;
c. allowing the reaction of step b to proceed for a period of time at room temperature;
d. adding said quantity of suitable substrate for deoxofluorination;
e. heating mixture to reflux for until completion.

13. The method of claim 12 wherein said excess quantity of dried alkali metal fluoride, said quantity of mesityl disulfide, said excess quantity of bromine, and said quantity of suitable substrate for deoxofluorination are, respectively, six equivalents of dried alkali metal fluoride, one equivalent of mesityl disulfide, three equivalents of bromine, and 0.75 equivalents of suitable substrate for deoxofluorination.

14. The method of claim 13 wherein said quantity of suitable substrate for deoxofluorination is an aldehyde or ketone, and said quantity of dried alkali metal fluoride is dried potassium fluoride.

15. The method of claim 14 wherein said quantity of aldehyde or ketone is selected from the group comprising of: Benzaldehyde, p-Bromobenzaldehyde, p-Tolualdehyde, Acetophenone, 2-Butanone, or Isobutyraldehyde, and wherein said quantity of suitable solvent is selected from the group comprising anhydrous acetonitrile and propionitrile.

16. The method of claim 14 wherein the reaction of step b comprises the sub-steps of: first, placing said quantity of suitable solvent under nitrogen, second, stirring in said excess quantity of dried potassium fluoride, third, adding said quantity of mesityl disulfide, fourth, cooling the mixture to 0° C., and fifth, adding said bromine, dropwise.

17. A method of preparing a compound of the formula (I) 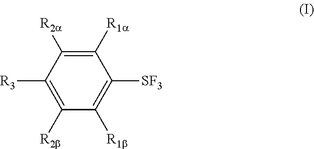
comprising the step of reacting a compound of formula (Ia) with a quantity of Bromine and an alkali metal fluoride, 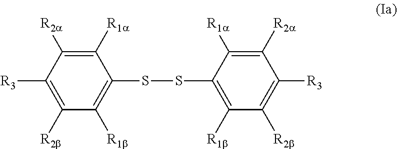
in which: R_{1α} and R_{1β} are independently a hydrogen atom or a primary or secondary alkyl group having from one to eight carbon atoms; R_{2α} and R_{2β} are independently a hydrogen atom or a primary, secondary, or tertiary alkyl group having from one to eight carbon atoms;
R₃ is a hydrogen atom, a halogen atom, or a primary, secondary, or tertiary alkyl group having from one to eight carbon atoms; wherein, when R₃ is a hydrogen atom, at least two of R_{1α}, R_{1α}, R_{2α}, and R_{2β} are primary, secondary, or tertiary alkyl groups having from one to eight carbon atoms and the others are a hydrogen atom, and wherein, when R₃ is a primary alkyl group having from one to eight carbon atoms, at least one of R_{1α}, R_{1β}, R_{2α}, and R_{2β} is a primary, secondary, or tertiary alkyl group having from one to eight carbon atoms and the others are a hydrogen atom, and wherein when at least two of R_{2α}, R_{2β}, and R₃ are tertiary alkyl groups, the tertiary alkyl groups are non-adjacent.

18. The method of claim 17 further comprising the step of introducing a suitable solvent and wherein said alkali metal fluoride is potassium fluoride or cesium fluoride.

19. The method of claim 18 wherein said suitable solvent is acetonitrile.

20. The method of claim 17 wherein the compound that is prepared is selected from the group consisting of: 2,6-dimethyl-4-tert-butylphenylsulfur trifluoride; 4-tert-butylphenylsulfur trifluoride; 2,4,6-trimethylphenylsulfur trifluoride; 2,4-dimethylphenylsulfur trifluoride; 2,5-dimethylphenylsulfur trifluoride; 2,6-dimethylphenylsulfur trifluoride; 4-fluorophenylsulfur trifluoride; 4-chlorophenylsulfur trifluoride; 3-methyl-4-chlorophenylsulfur trifluoride; and 2,4,6-tri(isopropyl)phenylsulfur trifluoride.

21. A method of preparing a compound of formula (II): 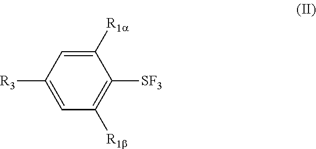
comprising the step of reacting a compound of formula (IIa) with Bromine and an alkali metal fluoride, 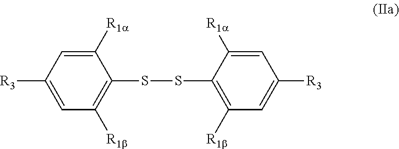
in which: R_{1α} and R_{1β} are independently a hydrogen atom or a primary or secondary alkyl group having from one to eight carbon atoms; R₃ is a hydrogen atom, a halogen atom, or a primary, secondary, or tertiary alkyl group having from one to eight carbon atoms; wherein when R₃ is a hydrogen atom, R_{1α} and R_{1β} are independently a primary or secondary alkyl group having from one to eight carbon atoms, and wherein, when R₃ is a primary alkyl group having one to eight carbon atoms, at least one of R_{1α} and R_{1β} is a primary or secondary alkyl group having from one to eight carbon atoms and the other is a hydrogen atom.

22. The method of claim 20 further comprising the step of introducing a suitable solvent and wherein said alkali metal fluoride is potassium fluoride or cesium fluoride.

23. The method of claim 21 wherein said suitable solvent is acetonitrile.

24. A method of in situ deoxofluorination comprising the steps of:
preparing a fluorinating agent of formula (I), and 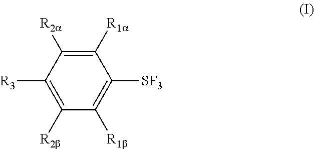
introducing a target compound having one or more oxygen or oxygen-containing groups under conditions that allow for one or more fluorine atoms to be introduced into the target compound.

25. The method of claim 24 wherein said step of preparing a fluorinating agent of formula (I) further comprises the sub-step of: reacting a compound of formula (Ia) with a quantity of Bromine and an alkali metal fluoride, 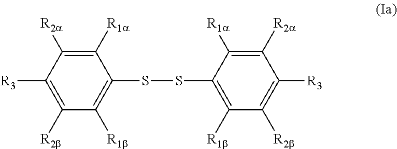
in which: R_{1α} and R_{1β} are independently a hydrogen atom or a primary or secondary alkyl group having from one to eight carbon atoms; R_{2α} and R_{2β} are independently a hydrogen atom or a primary, secondary, or tertiary alkyl group having from one to eight carbon atoms; R₃ is a hydrogen atom, a halogen atom, or a primary, secondary, or tertiary alkyl group having from one to eight carbon atoms; wherein, when R₃ is a hydrogen atom, at least two of R_{1α}, R_{1β}, R_{2α}, and R_{2β} are primary, secondary, or tertiary alkyl groups having from one to eight carbon atoms and the others are a hydrogen atom, and wherein, when R₃ is a primary alkyl group having from one to eight carbon atoms, at least one of R_{1α}, R_{1β}, R_{2α}, and R_{2β} is a primary, secondary, or tertiary alkyl group having from one to eight carbon atoms and the others are a hydrogen atom, and wherein when at least two of R_{2α}, R_{2β}, and R₃ are tertiary alkyl groups, the tertiary alkyl groups are non-adjacent.

26. The method of claim 24 wherein said target compound is any suitable substrate for deoxofluorination and wherein the fluorinating agent is selected from the group consisting of 2,6-dimethyl-4-tert-butylphenylsulfur trifluoride; 4-tert-butylphenylsulfur trifluoride; 2,4,6-trimethylphenylsulfur trifluoride; 2,4-dimethylphenylsulfur trifluoride; 2,5-dimethylphenylsulfur trifluoride; 2,6-dimethylphenylsulfur trifluoride; 4-fluorophenylsulfur trifluoride; 4-chlorophenylsulfur trifluoride; 3-methyl-4-chlorophenylsulfur trifluoride; and 2,4,6-tri(isopropyl)phenylsulfur trifluoride.

27. A method of in situ deoxofluorination comprising the steps of:
preparing a fluorinating agent of formula (II), and 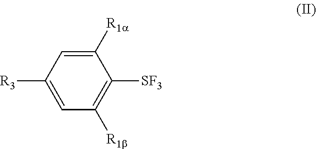
introducing a target compound having one or more oxygen or oxygen-containing groups under conditions that allow for one or more fluorine atoms to be introduced into the target compound.

28. The method of claim 27 wherein said step of preparing a fluorinating agent of formula (II) further comprises the sub-step of: reacting a compound of formula (IIa) with a quantity of Bromine and an alkali metal fluoride, 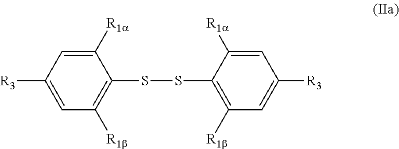
in which: R_{1α} and R_{1β} are independently a hydrogen atom or a primary or secondary alkyl group having from one to eight carbon atoms; R₃ is a hydrogen atom, a halogen atom, or a primary, secondary, or tertiary alkyl group having from one to eight carbon atoms; wherein when R₃ is a hydrogen atom, R_{1α} and R_{1β} are independently a primary or secondary alkyl group having from one to eight carbon atoms, and wherein, when R₃ is a primary alkyl group having one to eight carbon atoms, at least one of R_{1α} and R_{1β} is a primary or secondary alkyl group having from one to eight carbon atoms and the other is a hydrogen atom.

29. The method of claim 28 wherein said target compound is any suitable substrate for deoxofluorination.
